(19) United States
(12) Patent Application Publication
(10) Pub. No.: US 2009/0192428 A1
(43) Pub. Date: Jul. 30, 2009

(54) SOFT WRAP FOR TREATING PLANTAR FASCIITIS

(75) Inventors: MARY DeBOER, ARNOLDS PARK, IA (US); TERRY KOUNKEL, SPIRIT LAKE, IA (US); IVAN E. BROWN, SPIRIT LAKE, IA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, SPIRIT LAKE, IA (US)

(21) Appl. No.: 12/022,657

(22) Filed: Jan. 30, 2008

(51) Int. Cl. A61F 5/00; A61F 13/00

(52) U.S. Cl. 602/27; 602/65

(57) ABSTRACT

An adjustable soft wrap for treating plantar fasciitis is provided. A soft wrap is cut from material in such a manner to allow independent compression adjustment on the ankle and the middle of the foot while simultaneously having straps to allow tension adjustment of the stretch tension on the planter fascia.

### FIELD OF THE INVENTION

The present invention relates to a soft wrap for treating plantar fasciitis.

### BACKGROUND OF THE INVENTION

Human injuries resulting from accidents and occupational and recreational physical activities are frequent. Such injuries and consequent pain frequently result from overloading, repetitive strain and other causes resulting in injuries to muscles, tendons, ligaments and other body tissue. Such injuries result not only in pain but also in the need to limit or eliminate physical activity involving the injured area, with resulting loss of time, diminished work, and diminished recreational activities.

Plantar fasciitis is one of the most common causes of heel pain. This condition occurs in a wide variety of individuals. Commonly, age at onset is in the mid-40's, but plantar fasciitis can develop at any age, particularly in young athletes who put extreme exertion on their feet such as basketball and volleyball players. Many studies have shown a female-male predominance of 3:1. Many patients with plantar fasciitis also have moderate pronation; about 15% have high-arched, ridge foot; and the remainder have an anatomically normal or non-affected foot. Some 45% of the patients who undergo radiography for suspected plantar fasciitis are found to have a subcalcaneal or “bone” spur.

Evidence of the need for effective therapy is apparent when it is considered that over 95% of all heel pain is diagnosed as plantar fasciitis. Plantar fasciitis is best described as an inflammation of the ligament that runs from the heel to the ball of the foot, which helps support the arch. Patients with plantar fasciitis will experience pain, upon standing, on the bottom or inside of their heel. Typically, the pain is worse in the morning when getting out of bed and after resting when the person stands up, putting stretching pressure on the tendons.

Typically the primary anatomic cause of plantar fasciitis is some degree of microtrauma and tearing at the site of the Plantar Fascia insertion. These abnormalities, which may also be present at the origin of the Plantar Fascia, result from repetitive trauma and collagen degeneration and angiofibroblastic hyperplasia. Upon physical examination the range of motion of the affected ankle is less than that of the contralateral ankle. By pressing the thumb against the middle of the affected heel, the physician can delineate the area of the Plantar Fascial pain. Pressure similarly applied underneath the calcaneus reveals the area of subcalcaneal pain. The correlation between plantar fasciitis and subcalcaneal spurs is not significant, therefore radiographic findings are not specific. Conservative treatment, including night splints, results in relief of plantar fasciitis in 85% of patients. In 15% of patients in whom this approach fails, surgery is indicated.

One medical method known in the art in reducing Plantar Fascial pain is to stretch the Plantar Fascia for a period of time. By keeping the Plantar Fascia on stretch, it is believed that an ultimate reduction of the internal tension of the Plantar Fascia can be achieved. Through this treatment, it is believed that the pain associated with this medical condition can be reduced, and possibly eliminated.

A typical treatment program would have the patient wear the splint while sleeping, and remove the splint immediately upon awakening in the morning. The patient will continue wearing the night splint for a 3-month period. After that time the patient will be weaned off of the splint in 2-week increments, using the device every other night, then every third night, then every fourth night, and from then on as needed.

A number of plantar fasciitis night splints are known in the art. One successful unit is commonly assigned U.S. Pat. No. 6,267,742. There are many others that have been patented. However, none have the suspension architecture or offer the comfort, ease of use, compactability, or degrees of rotation and angulation of U.S. Pat. No. 6,267,742.

By way of illustration of the splint state of the art, U.S. Pat. No. 4,649,939, issued to R. Curtis on Mar. 17, 1987 utilized over a shoe. U.S. Pat. No. 5,038,762, issued to H. Hess, et. al., on Aug. 13, 1991, teaches of a U-Shaped yoke which can be wrapped about the heel and ankle. U.S. Pat. No. 5,090,404, issued to C. Kallassy on Feb. 25, 1992, teach of another way to place a strap about the heel and foot. U.S. Pat. No. 5,257,969 issued to C. Mance on Nov. 2, 1993 teaches of a foot support which consists of a toe pouch and straps the wrap about the ankle. U.S. Pat. No. 5,425,701 issued to C. Oster et al. on Jun. 20, 1995, teaches of a boot with upright struts which attach to a foot pad designable for each patient's foot shape. U.S. Pat. No. 5,472,411 issued to H. Montag, et. al., on Dec. 5, 1995 teaches of a U-shaped flexible joint collar which wraps about the foot, heel and ankle.

Another type of orthotic device is a soft wrap for support. U.S. Pat. No. 5,620,413, issued to D. Olson on Jul. 14, 1995 teaches of an ankle brace and wrap comprised of a support sleeve to fit over the foot. U.S. Design Pat. No. Des. 388,174 issued to W. Stano on Dec. 23, 1997 teaches of an ankle brace which wraps about the lower leg, ankle, and foot. U.S. Pat. No. 5,645,525, issued to R. Krivosha on Jul. 8, 1997 teaches of a heel stabilizing device which fits over the foot and heel. While these devices teach a flexible means of foot support, they do not teach a soft wrap means to keep the foot on stretch which would properly facilitate the treatment indicated for a diagnosis of plantar fasciitis, and at the same time allow pressure adjustment of the soft wrap on the ankle and foot.

Olson U.S. Pat. No. 5,620,413 does teach a soft wrap with straps, but it is an ankle compression wrap, not allowing any pressure adjustment on the ankle or foot. And it is designed for Achilles tendon treatment, not plantar faciitis. Another commercially successful soft wrap is Thermoskin® Plantors FXT, which uses a sock with a strap from the toe to ankle front to keep the plantar fascia on stretch. This can and does cause toe pain.

There is therefore a continuing need to develop units which provide doctors with choices to allow balancing of cost, convenience, and adjustments to allow maximum flexibility to fit individual patient needs.

In summary, while soft wrap night splints have been used in the past for plantar fasciitis, all that are used are deficient in either comfort, suspension architecture, adjustability, or ease of patient use.

Accordingly a primary objection of the present invention is to provide a soft wrap for treating plantar fasciitis that allows independent compression adjustment on the ankle, and the middle of the foot, while simultaneously allowing full flexible tension adjustment to stretch the plantar fascia.

It is a further objective of the invention to provide a soft wrap meeting the above objective wherein the wrap can be made from a single piece of wrap material.

Yet a further objective of the present invention to provide a wrap of a material which has a covering cloth to allow hook and pile fasteners for straps and therefore is complete adjustability without having mechanical strap fasteners.

Yet a further objective of the invention is to provide all of the above features in a soft wrap that does not use any rigid shell, making it less expensive to manufacture and fully flexible and adjustable.

The method and manner of accomplishing these and other objectives is described below.

### BRIEF SUMMARY OF THE INVENTION

An adjustable soft wrap for treating plantar fasciitis is provided. A soft wrap is cut from material in such a manner to allow independent compression adjustment on the ankle and the middle of the foot while simultaneously having straps to allow tension adjustment of the stretch tension on the planter fascia.

### BRIEF DESCRIPTION OF THE DRAWINGS

FIG. 1 is a perspective view of the unit of the present invention on a wearer's foot.

FIG. 2 is an inside layout of the wrap.

FIG. 3 is an outside layout of the wrap.

FIG. 4 is a sectional view through the wrap showing the embedded plate.

### DETAILED DESCRIPTION OF THE PREFERRED EMBODIMENT

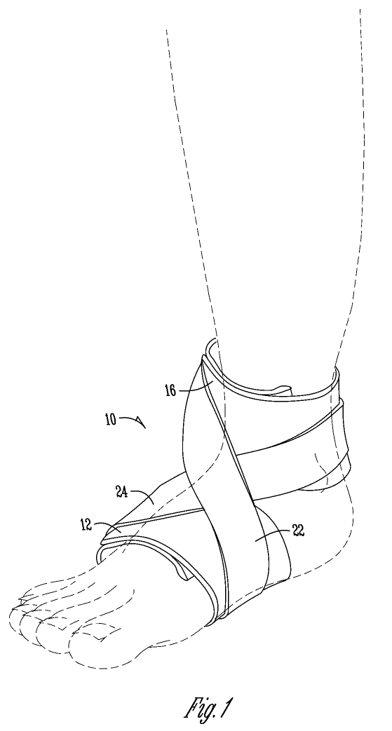

FIG. 1 shows the adjustable soft wrap 10 placed on a wearer's foot with tension straps adjusted to provide stretch tension to the plantar fascia.

The foot wrap 10 is made of a soft, durable natural fiber or foamed polymeric material designed to encompass a portion of the foot, the ankle and lower leg. Preferably, the wrap is made so that the anterior of the portion which covers the leg and the top of the portion which covers the foot can be opened, adjusted and then closed and secured. Preferably, these means of securing the wrap include covering cloth useable with hook and pile fasteners sold under the trademark “Velcro”.

The use of the exterior covering cloth that is useable with hook and pile fastener means that the tension straps (explained below) do not need to have any adjustable mechanical hardware to be fully adjustable. This will maximize patient comfort.

As used herein the term thistle cloth refers to a foam/fabric laminate with a hook compatible fabric that serves as the outer covering.

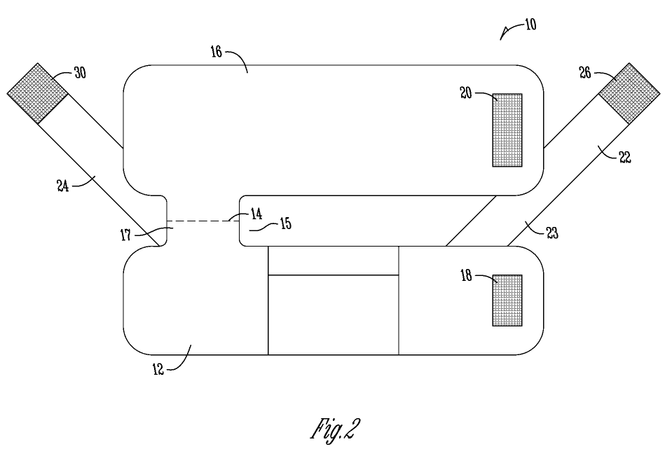

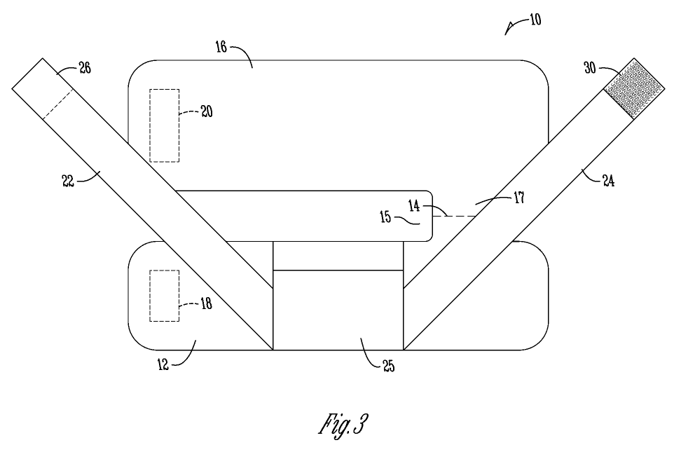

FIG. 2 shows the inside layout of the soft wrap 10; FIG. 3 shows the outside layout of the soft wrap 10.

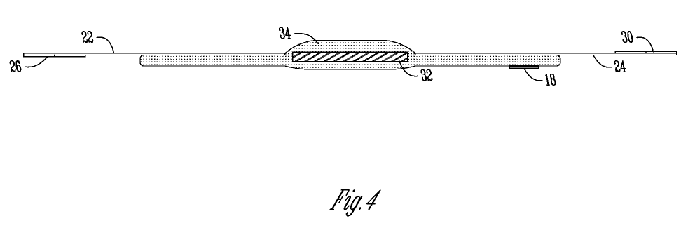

As shown in FIG. 2, the soft wrap 10 can be cut from a single piece of cloth of the type above described. The configuration shown in FIG. 2, is the inside (i.e., the side that will be against the foot). As seen, it is comprised of a bottom panel 12, a top panel 16, divided by a cutout 15 and held together by connector 17 portion along fold line 14. Bottom panel 12 has a hook patch 18 and top panel 16 has a corresponding hook patch 20. Attached to bottom panel 12 and sewn in are straps which can be woven straps, referred to herein as tension strap end portion 22 and end portion 24, each of which has at its terminal end patches 26 and 30 respectively. As seen in FIG. 4, panel 14 has an interior embedded foot plate 32 that fits under the foot, in the area of the arch and a cushion 34 over it embedded in the cloth wrap 10. The foot plate 32 can be a rigid plastic material such as High Density Polyethylene.

The operation of the unit works as follows. Looking at FIG. 2 particularly. The patient places his or her foot on top of cushion 34 of bottom panel 12. It is folded up along the ankle fold line 14, wrapped around the ankle. Hook pile 20 attaches to the exterior side of the cloth wrap 10 as tight as one wishes depending upon where the hook patch is attached. Similarly one hook panel 18 is wrapped around the mid foot and hook patch 18 is attached to the wrap to independently adjusted tension. Top panel 16 and bottom panel 12 are therefore independently wrapped and adjusted for compression around the ankle in the front of the foot.

Tension strap ends 22 and 24 of strap 23 are wrapped and crossed as shown in FIG. 1. The strap 23 is secured to the bottom of the panel (FIG. 3) with a hook patch 25. This allows the tension strap 23 to be positioned forward or backward under the forefoot for universal sizing comfort and effectiveness. The stretching tension against the plantar fascia is adjusted depending upon where patches 26 and 30 are attached to the exterior of top panel 16.

It can therefore be seen that complete and independent adjustability are provided for the wrap around the ankle and the wrap around the front of the foot offering 100% compression adjustment. Similarly tension strap ends 22 and 24 can be completely adjusted to provide whatever tension one desires on the plantar fascia, and this is done without having any mechanical contrivances attached to the straps. As a result for this maximized and adjustability is also maximized. This adjustability and maximized comfort has never been achieved in a soft wrap plantar fascia orthotic before.

It can therefore be seen that the invention accomplishes at least all of its stated objectives.

1. An adjustable soft wrap for treating plantar fasciitis, comprising:
a soft flexible foot wrap having an ankle portion and a middle foot portion which allows independent compression adjustment on the ankle portion and the middle of the foot portion; and
a fully adjustable tension strap having two ends, one attached to the ankle portion and the other to wrap around and attach to the foot portion to allow adjustable stretch tensioning of the plantar fascia.

2. The soft wrap of claim 1 wherein the two ends have hook patches for securement to the soft wrap.

3. The soft wrap of claim 1 which is made from thistle cloth using hook and pile fasteners for attachment.

4. The adjustable soft wrap of claim 1 wherein the wrap is made from a single piece cutout.

5. The soft wrap of claim 1 which uses an embedded and cushioned plate.

6. The soft wrap of claim 1 wherein the tension strap is secured to the exterior side of the soft wrap with a hook patch.

7. The soft wrap of claim 6 wherein there is a second tension strap attached to the exterior side of the soft wrap with a hook patch.
